(19) United States
(12) Patent Application Publication
(10) Pub. No.: US 2025/0312455 A1
(43) Pub. Date: Oct. 9, 2025

(54) PLASMONIC METAL-COLLAGEN HYBRID NANOPARTICLES FOR PROTEIN DELIVERY AND METHOD OF PREPARATION THEREOF

(71) Applicant: University of Seoul Industry Cooperation Foundation, Seoul (KR)

(72) Inventors: Inhee CHOI, Seoul (KR); DoYun KIM, Seoul (KR); Seungki LEE, Seoul (KR)

(73) Assignee: University of Seoul Industry Cooperation Foundation, Seoul (KR)

(21) Appl. No.: 19/097,845

(22) Filed: Apr. 1, 2025

(30) Foreign Application Priority Data
Apr. 5, 2024 (KR) 10-2024-0046397

(51) Int. Cl. A61K 41/00; A61K 38/18; A61K 38/44; A61K 47/65; A61K 47/69

(52) U.S. Cl. CPC A61K 41/0057; A61K 38/1808; A61K 38/446; A61K 47/65; A61K 47/6903; C12Y 115/01001

(57) ABSTRACT

The present invention relates to plasmonic metal-collagen hybrid nanoparticles that have excellent protein loading efficiency and excellent biocompatibility and can be utilized as protein delivery vehicles. In the present invention, a plasmonic metal-collagen hybrid nanoparticle for protein delivery, comprising a collagen of 1 kDa to 30 kDa, a thermosensitive polymer monomer, a plasmonic metal nanoparticle, and a photoinitiator, and a method for preparing the same are provided.

### CROSS-REFERENCE TO RELATED APPLICATIONS

This patent application claims the benefit of priority from Korean Patent Application No. 10-2024-0046397, filed on Apr. 5, 2024, the contents of which are incorporated herein by reference.

### BACKGROUND OF THE INVENTION

### 1. Field of the Invention

The present invention relates to a plasmonic metal-collagen hybrid nanoparticle for protein delivery and a method for preparing the same.

### 2. Description of the Related Art

A hydrogel is a type of cross-linked macromolecule with a gel-like structure inside. In an aqueous medium, hydrogels can exhibit reversible swelling/deswelling behavior in response to the surrounding environment or stimuli. Recently, methods for synthesizing these hydrogels not only in the form of films but also in the form of spherical nanoparticles of micrometer size (microgels) or submicrometer size (nanogels) have been reported. An important means of functionalizing these hydrogels is to insert stimuli-sensitive moieties into them. For example, the combination of a polymer or polymer component that is sensitive to stimuli such as heat or light, and a metal component that has optical properties arising from unique quantum-scale effects, forms a hybrid gel with novel properties. These new types of hybrid gels have attracted great attention, and their potential applications in various fields such as catalysis, molecular detection, and drug delivery have been extensively studied.

In the prior art, patent reference 1 (Korean Patent No. 10-2221924) discloses a method for preparing hybrid gel particles in which plasmonic gold nanoparticles are spontaneously embedded. Hybrid gel particles can induce structural changes in response to heat and/or light, and molecules loaded in the polymer network of these hybrid gel particles can be utilized as drug delivery vehicles that are released in response to heat and/or light.

However, in the case of hybrid gel particles using the linker molecule disclosed in patent reference 1 (Korean Patent No. 10-2221924), although they are useful as low-molecular compound delivery vehicles, improvement in loading efficiency was required for protein delivery.

### BRIEF SUMMARY OF THE INVENTION

It is an object of the present invention to provide a hybrid nanoparticle suitable for protein delivery into a living body.

It is another object of the present invention to provide a method for preparing a hybrid nanoparticle suitable for protein delivery into a living body.

To achieve the above objects, in an aspect of the present invention, the present invention provides a plasmonic metal-collagen hybrid nanoparticle for protein delivery, comprising a collagen of 1 kDa to 30 kDa, a thermosensitive polymer monomer, a plasmonic metal nanoparticle, and a photoinitiator.

In another aspect of the present invention, the present invention provides a protein delivery vehicle comprising a plasmonic metal-collagen hybrid nanoparticle.

In addition, the present invention provides a method for preparing a plasmonic metal-collagen hybrid nanoparticle for protein delivery, comprising a step of irradiating a mixture containing collagen of 1 kDa to 30 kDa, a thermosensitive polymer monomer, a plasmonic metal precursor, and a photoinitiator with ultraviolet light.

### Advantageous Effect

The plasmonic metal-collagen hybrid nanoparticle for protein delivery provided in one aspect of the present invention has a relatively high protein loading efficiency and excellent biocompatibility, and thus can be utilized as a protein delivery vehicle.

### BRIEF DESCRIPTION OF THE DRAWINGS

FIG. 1 is a graph showing the comparison of the EGF protein loading efficiency of nanoparticles made using various linker molecules.

FIG. 2 is a graph showing the size and polydispersity of CHP formed according to the concentration of Au ions.

FIG. 3 is a graph showing the average hydrodynamic diameter of CHP depending on the presence or absence of AuNP introduction.

FIG. 4 shows the SEM-EDS data of Au-CHP.

FIG. 5A shows the C₁ₛXPS analysis data of Au-CHP.

FIG. 5B shows the N₁ₛXPS analysis data of Au-CHP.

FIG. 5C shows the O₁ₛXPS analysis data of Au-CHP.

FIG. 5D shows the Au_{4f}XPS analysis data of Au-CHP.

FIG. 6A is a set of photographs showing the turbidity of Au-CHP according to temperature.

FIG. 6B is a graph showing the transmittance of CHP and Au-CHP according to temperature.

FIG. 7A shows the ¹H NMR analysis data of the PNIPAM terminus of Au-CHP.

FIG. 7B shows the ¹H NMR analysis data of the PNIPAM side chain of Au-CHP.

FIG. 8A shows the temperature evolution data when Au-CHP and CHP were irradiated with a laser at 532 nm.

FIG. 8B is a graph showing the temperature change over time after laser irradiation of Au-CHP and CHP.

FIG. 9 is a graph showing the relative diameter according cycle, when exposed to light or directly heated.

FIG. 10 is a graph showing the loading efficiency of Au-CHP by protein type.

FIG. 11 is a graph showing the change in surface charge of Au-CHP before and after protein loading.

FIG. 12 shows the cumulative protein release comparison data of Au-CHP and CHP after light illumination.

FIG. 13 is a graph showing the cumulative release amount of Au-CHP loaded with each protein over time (in hours), depending on the presence or absence of light illumination.

FIG. 14 is a graph showing the cumulative release amount of each protein of Au-CHP loaded with each protein over time (in days) irradiated with light in FIG. 13.

FIG. 15 is a graph showing the cumulative release amount of protein from Au-CHP at room temperature and when exposed to 38° C. (temperature according to photothermal effect) for 30 minutes.

FIG. 16A is a set of fluorescence images of HDF cells with and without Au-CHP upon FGF2 delivery.

FIG. 16B is a graph showing the uptake rate of HDF cells with and without Au-CHP upon FGF2 delivery.

FIG. 17A is a set of images of HDF cells showing the results of wound healing analysis over time.

FIG. 17B is a graph showing the relative recovery area of HDF cells according to the treated material.

FIG. 17C shows the cell front velocity data.

FIG. 18A is a set of phase contrast images and fluorescent images of HDF cells depending on the use of Au-CHP and the presence or absence of light during SOD1 delivery.

FIG. 18B is a graph showing the mean fluorescence intensity (MFI) per cell of Hoechst and SOD1.

FIG. 19 is a set of fluorescent images of reactive oxygen species in HDF cells depending on the use of Au-CHP and the presence or absence of light during SOD1 delivery.

FIG. 20 is a graph showing the active oxygen scavenging rate (scavenging %) depending on the use of Au-CHP and the presence or absence of light.

FIG. 21A is a confocal image of EGF protein and a fluorescence intensity profile of the white cross-section line in the image. The dashed line indicates the depth of confocalization between Hoechst staining (i.e. nuclei) and Alexa 488 (i.e. EGF).

FIG. 21B is a confocal image of EGF-loaded Au-CHP and a fluorescence intensity profile of the white cross-section line in the image.

FIG. 21C is a confocal image when light is irradiated on EGF-loaded Au-CHP and a fluorescence intensity profile of the white cross-section line of the image.

FIG. 22A is a set of fluorescent images of spheroids depending on the presence or absence of EGF-loaded Au-CHP treatment and time.

FIG. 22B is a graph showing the cumulative MFI of EGF protein delivery of spheroids.

FIG. 22C is a graph showing the diameter of spheroids treated with EGF protein-loaded Au-CHP over time.

FIG. 23 is a graph showing the cell viability (%) upon treatment with Au-CHP.

FIG. 24A is a set of images of hemolysis of red blood cells according to treatment with different concentrations of Au-CHP or gold nanoparticles.

FIG. 24B is a graph showing the hemolysis (%) of red blood cells according to treatment with different concentrations of Au-CHP or gold nanoparticles.

### DETAILED DESCRIPTION OF THE INVENTION

Hereinafter, the present invention is described in detail.

The embodiments of this invention can be modified in various other forms, and the scope of the present invention is not limited to the embodiments described below. It is well understood by those in the art who has the average knowledge on this field that the embodiments of the present invention are given to explain the present invention more precisely.

In addition, the “inclusion” of an element throughout the specification does not exclude other elements, but may include other elements, unless specifically stated otherwise.

In an aspect of the present invention, a plasmonic metal-collagen hybrid nanoparticle for protein delivery, comprising a collagen of 1 kDa to 30 kDa, a thermosensitive polymer monomer, a plasmonic metal nanoparticle, and a photoinitiator is provided.

In this specification, collagen is a biological polymer that constitutes major body organelles such as the extracellular matrix, and is composed of a biological structure suitable for interaction with internal components. Specifically, collagen chains contain glycine (Gly) at every third residue, which creates a structure with characteristic repeat sequences and many residues to which external peptides or enzymes can be attached. In addition, hydroxyproline (Hyp), which is very high in content like glycine, increases structural stability while exposing hydrogen bonding sites to the outside, thereby inducing binding to other external peptides.

In general, collagen contains cations and thus has a positive surface charge.

The structures of glycine and hydroxyproline are shown in the following formulas 1 and 2.

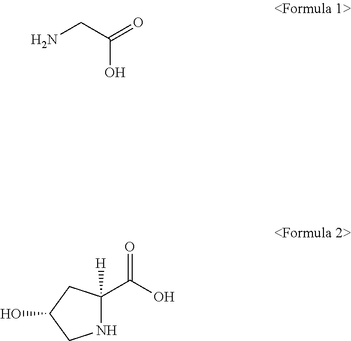 [SMILES: NCC(=O)O ; O=C(O)[C@@H]1C[C@@H](O)CN1]

Meanwhile, during the process of collagen being naturally degraded in the body, it is resistant to common proteases that break down the loaded proteins together due to the stability derived from its unique structure. Since collagen is composed of a stable triple helix structure, it is degraded only by specific enzymes of the matrix metalloproteinase (MMP) family rather than general proteases, and can be degraded safely and quickly in the body while protecting the loaded proteins. Accordingly, collagen may be used to impart biocompatibility.

The commonly used porcine-derived collagen has an average molecular weight of 300 kDa and a dense structure. Therefore, when producing conventional nanocarriers using collagen, there is a limit to size control, so a purification process to gelatin, which is a peptide unit, is required.

On the other hand, marine collagen, which is derived from jellyfish or seaweed, has an average molecular weight of 3 kDa and has higher flexibility and exposure to molecular binding sites than general collagen. In addition, when forming a hydrogel structure with small molecular weight collagen, the size can be adjusted to enable cell penetration, and the polymer network becomes loose, allowing smooth drug loading. Therefore, in this specification, it is preferable to use marine-derived collagen.

In the present invention, collagen having a molecular weight of 1 kDa to 30 kDa or 2 kDa to 20 kDa, preferably 3 kDa to 10 kDa can be used.

In the present invention, a thermosensitive polymer monomer can be used to release a protein at a desired location. The thermosensitive polymer monomer that can be used herein includes N-isopropylacrylamide (NIPAM), N-vinylcaprolactam, (dimethylamino)-ethylmethacrylate, and N, N-dimethylacrylamide.

While most polymers increase in solubility with increasing temperature, thermosensitive polymers, which exhibit a low critical solution temperature (LCST), have the property of decreasing solubility with increasing temperature (shrinking at temperatures above LCST). Thermosensitive polymers have hydrophobic groups in common, and in response to a change in temperature, water molecules bound to these hydrophobic groups are released, causing phase separation and resulting in shrinkage. When the LCST is close to the body temperature, at room temperature below the LCST, the thermosensitive polymer exists in an aqueous solution state, and near the body temperature, the thermosensitive polymer becomes a gel due to the strengthened hydrophobic interaction and shrinks to release the loaded drug.

In the present invention, plasmonic metal nanoparticles can be included so as to release the loaded protein by increasing the temperature of the thermosensitive polymer. Structural changes in plasmonic metal-collagen hybrid nanoparticles are induced by utilizing the photothermal effect induced when irradiated with light of a wavelength absorbed by plasmonic metal nanoparticles.

The plasmonic metal nanoparticles can generate a photothermal effect and can be selected from gold, silver, platinum, aluminum, or copper, and in a specific embodiment of the present invention, gold was used.

In the present invention, the plasmonic metal nanoparticles may be included in an amount of 1 to 40 volume %, 1 to 30 volume %, or 1 to 20 volume %, and the value of the temperature increase due to the photothermal effect can be controlled by regulating the ratio of the nanoparticles.

The plasmonic metal nanoparticles have a nanoscale size, and may have, for example, a size of 1 nm to 100 nm, 1 nm to 50 nm, 1 nm to 30 nm, 1 nm to 20 nm, 1 nm to 10 nm, 2 nm to 9 nm, 3 nm to 8 nm, or 4 nm to 7 nm.

In the present invention, the protein to be loaded is not particularly limited in molecular weight and may have a molecular weight of, for example, 1 kDa to 1000 kDa, 1 kDa to 500 kDa, 1 kDa to 300 kDa, 1 kDa to 100 kDa, 10 kDa to 1000 kDa, 10 kDa to 500 kDa, 10 kDa to 300 kDa, or 10 kDa to 100 kDa, and generally has an anionic surface charge. Therefore, the protein can interact with collagen having a positive surface charge, which is another component of the present invention, to further increase the loading efficiency.

In another aspect of the present invention, a protein carrier comprising plasmonic metal-collagen hybrid nanoparticles according to the present invention is provided.

In another aspect of the present invention, a method for preparing a plasmonic metal-collagen hybrid nanoparticle for protein delivery, comprising a step of irradiating a mixture containing collagen of 1 kDa to 30 kDa, a thermosensitive polymer monomer, a plasmonic metal precursor, and a photoinitiator with ultraviolet light is provided.

As the plasmonic metal precursor, a metal salt can be used. The metal salt is a combination of metal cations and anions, and the metal in the metal salt can be gold (Au), silver (Ag), platinum (Pt), aluminum (AI), and copper (Cu). In the metal salt, the salt may be chloride, nitrate, carbonate, phosphate, borate, sulfate, oxide, sulfonate, stearate, myristate, acetate, acetylacetonate, a hydrate thereof, and a mixture thereof.

The plasmonic metal precursor may be included in the mixture solution in a concentration range of 0.001 mM to 5 mM, and 0.005 mM to 2 mM, but not always limited thereto.

In addition, 2-hydroxy-2-methylpropiophenone, 2-hydroxy-4′-(2-hydroxyethoxy)-2-methylpropiophenone, or 1-[4-(2-hydroxyethoxy)-phenyl]-2-hydroxy-2-methyl-1-propan-1-one can be used as the photoinitiator, either alone or in combination. As an example, 2-hydroxy-2-methylpropiophenone (Darocur®1173) can be used.

Hereinafter, the present invention will be described in detail by the following examples.

However, the following examples are only for illustrating the present invention, and the contents of the present invention are not limited thereto.

### Example 1: Exploration of Linker Molecules for Protein Loading

In order to find a linker molecule that is favorable for protein loading, hybrid gel particles with linker molecules bound thereto were prepared by referring to the preparation method of patent reference 1 (Korean Patent No. 10-2221924).

Specifically, a mixture containing 900 μL of a 2% (w/v) linker molecule solution, 900 μL of a 1% (w/v) NIPAM solution, 100 μL of distilled water (DI), and 100 μL of 5% (v/v) Darocur®1173 (photoinitiator) in 99% (v/v) ethanol was prepared. Then, the mixture was irradiated with ultraviolet light for 10 minutes to perform a radical reaction. The radical reaction was then terminated by adding 1 mL of distilled water, and particles with different linker molecules were obtained.

The linker molecules used and their molecular weights are shown in Table 1 below.

TABLE 1

| Linker molecule | Molecular weight (g/mol) |
|---|---|
| N,N-methylene bis(acrylamide) | 154.17 |
| Tryptophan | 204.23 |
| Sucrose | 342.3 |
| Alginic acid | 58,900 |
| Gelatin | 50,000-100,000 |
| Marine collagen | 3,000-10,000 |

For the particles prepared according to the linker molecules in Table 1 above, the particles prepared by using N,N-methylene bis(acrylamide) as a linker molecule are indicated as ‘M-PHN’, the particles prepared by using tryptophan as a linker molecule are indicated as ‘T-PHN’, the particles prepared by using sucrose as a linker molecule are indicated as ‘S-PHN’, the particles prepared by using alginic acid as a linker molecule are indicated as ‘A-PHN’, the particles prepared by using gelatin as a linker molecule are indicated as ‘G-PHN’, and the particles prepared by using the marine collagen of the present invention as a linker molecule are indicated as ‘Au-CHP’.

Then, protein loading was performed as follows to evaluate the protein loading efficiency of the particles according to the linker molecules.

The particles synthesized according to the linker molecules were loaded with epidermal growth factor (EGF) protein. First, the particles according to the linker molecules were dissolved in distilled water, to which protein (80 g/mL in NaH₂PO₄+NaOH buffer, pH 8.0) was added in a 1:9 ratio, followed by stirring in the dark for 24 hours. Afterwards, centrifugation was performed and the supernatant was collected to obtain the protein-loaded particles.

To measure the loading efficiency, Ara27 CPP conjugated protein (Ara27-EGF) was loaded and the protein loading efficiency was calculated.

### Experimental Example 1: Comparison of EGF Protein Loading Efficiency According to Linker Molecules

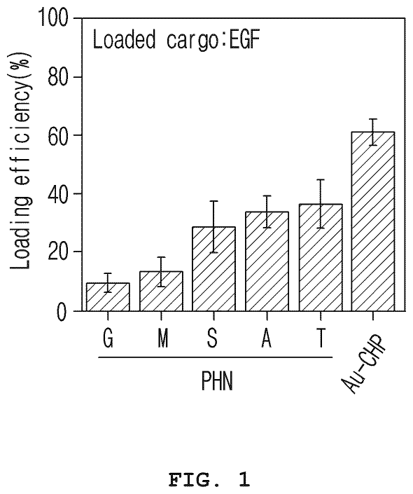

The EGF protein loading efficiency of the nanoparticles prepared using the linker molecules shown in Table 1 above was compared and presented in FIG. 1.

As a result, it was confirmed that the highest protein loading efficiency was achieved when collagen was used as a linker molecule. The molecular weight of the collagen used was approximately 3-10 kDa.

As the molecular weight of the polymer, which is the linker molecule used, decreased, the loading amount tended to increase, and it was confirmed that the protein loading efficiency of the nanoparticles prepared by using marine collagen, which is the linker molecule of the present invention, was the best compared to other linker molecules in Table 1 (60.86%). On the other hand, gelatin, a collagen polymer, has a very high molecular weight (50-100 kDa), so its polymer matrix is very dense and has many residues that help in protein loading, but its loading efficiency seems to be very low (G-PHN/9.35%).

As a result, it is expected that the protein loading efficiency will be the best when marine collagen with a molecular weight of 1000 to 30000 Da is used.

Hereinafter, in the present invention, marine collagen is used as a linker molecule, and the product is named CHP.

### Example 2: Preparation of Au-CHP

CHP containing gold nanoparticles was named Au-CHP.

In the preparation method of CHP according to Example 1, Au-CHP was prepared by introducing AuNP into CHP by adding 1 mM of HAuCl₄ instead of 100 μL of distilled water. The gold ions of HAuCl₄ bound to the hydroxyproline-rich residues of collagen and were reduced to AuNP in the collagen matrix (i.e., Au@Col). The photoinitiator, which dissociated during the polymerization process, simultaneously served as a reducing agent for gold ions and a polymerizing agent for hydrogel under ultraviolet light. The generated radicals are polymerized and act to induce collagen to be captured by PNIPAM (Poly(N-isopropylacrylamide)) due to the strong hydrogen bonding between the residual amide group of PNIPAM and the —COOH group of collagen.

### Experimental Example 2: Analysis of Size and Polydispersity According to Gold Precursor Concentration During Au-CHP Production

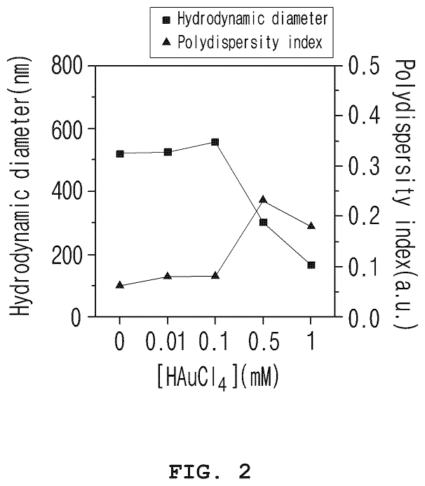

Au ions were spontaneously reduced by radicals to form AuNP inside CHP. The size and polydispersity of the CHP formed according to the concentration of Au ions were monitored (FIG. 2). In the preparation of Au-CHP, particles were prepared using different concentrations of gold precursor, i.e., 0, 0.01, 0.1, 0.1, 0.5, and 1 mM of gold precursor.

The use of HAuCl₄ above 0.5 mM resulted in drastic changes in both parameters, indicating that the AuNP in CHP limited the growth of the PNIPAM network.

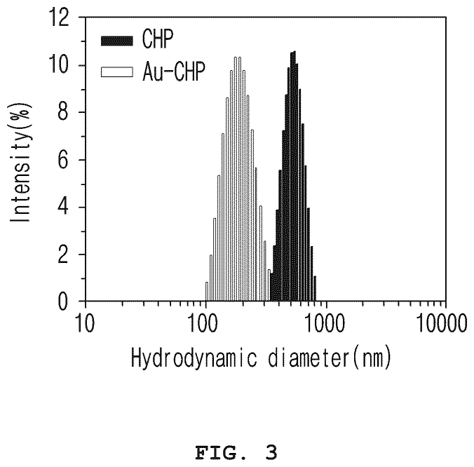

As shown in FIG. 3, the average diameter of CHP (558.69±130.67 nm) was significantly reduced in the presence of AuNP (195.77±62.25 nm for Au-CHP). Since natural collagen has negatively charged surface residues, adsorption of gold cations on collagen can neutralize the surface of the hydrogel formed. Then, the surface charge becomes positive depending on the PNIPAM coverage. This sequential change in the diameter and surface charge of CHP provides strong evidence for the synthesis procedure. The initial hydrodynamic diameter of Au-CHP was maintained for a long period of time (10 weeks), indicating excellent stability for long-term protein storage.

### Experimental Example 3: Analysis of Au-CHP Properties

### <3-1> SEM-EDS (SEM Energy-Dispersive X-Ray Spectroscopy) Analysis

SEM-EDS was performed to confirm that AuNP was loaded in CHP, and the results are shown in Table 2 and FIG. 4.

TABLE 2

| | Mass(%) | Atom(%) | 3σ(%) |
|---|---|---|---|
| C | 2.28 | 37.37 | 1.1 |
| N | 0.72 | 10.14 | 0.5 |
| O | 2.05 | 25.18 | 0.9 |
| Au | 27.31 | 27.30 | 3.1 |

### <3-2> XPS Analysis

X-ray photoelectron spectroscopy (XPS) was performed to determine the elemental composition and chemical valence state of Au-CHP.

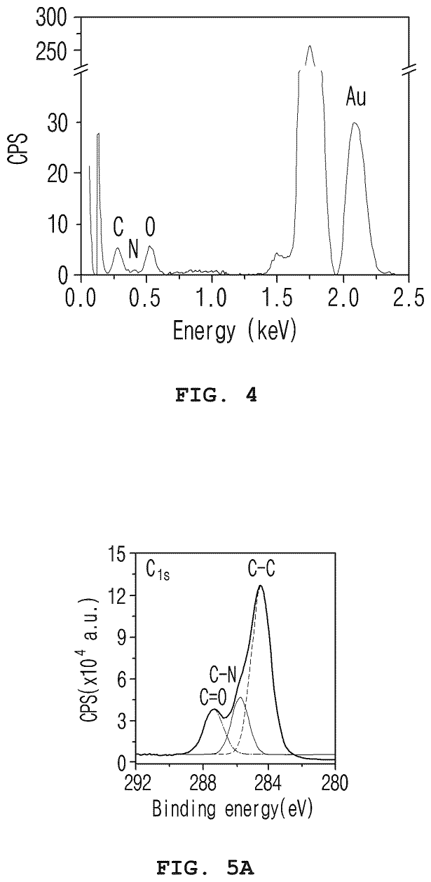

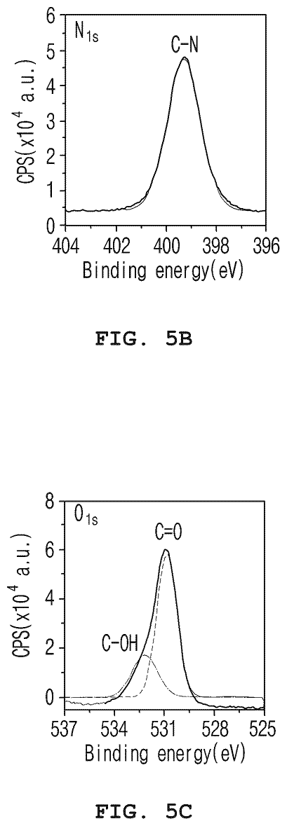

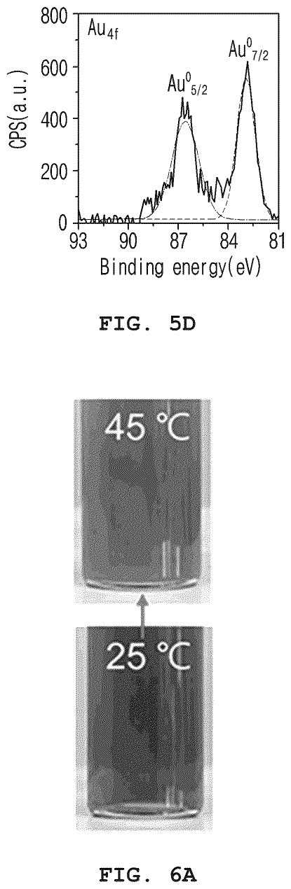

The narrow scan spectra of Au-CHP showed the peaks corresponding to C1s, O1s, N1 s, and Au4f at characteristic binding energies (FIG. 5A, FIG. 5B, FIG. 5C, and FIG. 5D, in that order, respectively).

In the C1s region of FIG. 5A, the spectrum was re-resolved into three peaks (284.49 eV for the C—C peak, 285.76 eV for the C—N peak, and 287.35 eV for the C═O peak) representing the PNIPAM-collagen hydrogel network. This was confirmed again by the observation of 531.04 eV for the C═O peak in the 01 s spectrum of FIG. 5C and 399.32 eV for the C—N peak in the Nis spectrum of FIG. 5B. The AuNP of Au-CHP was likely covered by a hydrogel network, and thus, the metallic Au⁰ peaks were observed with low intensity at 86.61 and 82.96 eV in FIG. 5D. In addition, the peak shifted to slightly higher binding energy, indicating that the AuNP was incorporated into the PNIPAM/collagen network through weak interactions. The PNIPAM network of Au-CHP can be transformed into flexible chains to globules by heating due to intermolecular aggregation under given conditions.

### Experimental Example 4: Analysis of Photothermal Properties of Au-CHP for Protein Delivery

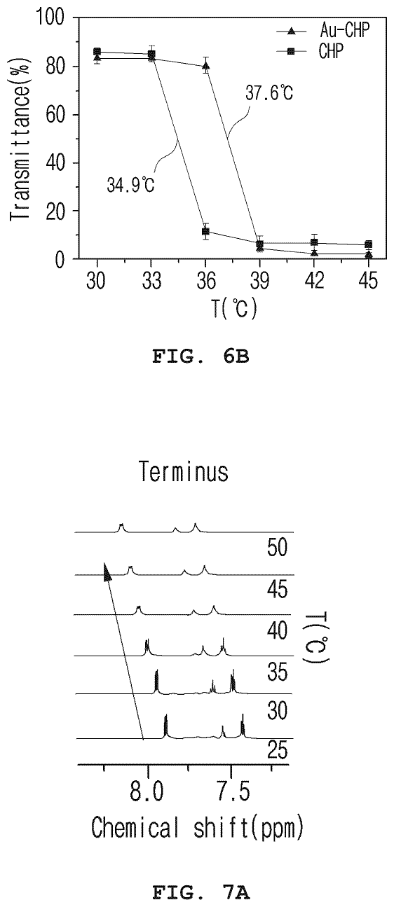

To investigate the heat-responsive properties, the turbidity of colloidal Au-CHP was monitored by increasing the solution temperature, and the photographs are shown in FIG. 6A and the transmittance graphs of CHP and Au-CHP are shown in FIG. 6B. As shown in FIG. 6B, the low critical solution temperature (LCST) of Au-CHP (approximately 37.6° C.) was higher than that of CHP (approximately 34.9° C.).

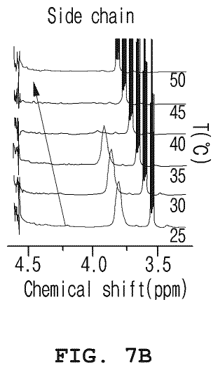

For further confirmation, ¹H NMR studies were performed to monitor the peak shifts of the polymer chains with increasing temperature (FIG. 7A, FIG. 7B). Both the terminal (benzophenone) and side chain (amide) of PNIPAM exhibited temperature-dependent peak shifts, indicating that the conformation changed by losing hydrogen bonds in the amide group of the PNIPAM network of Au-CHP.

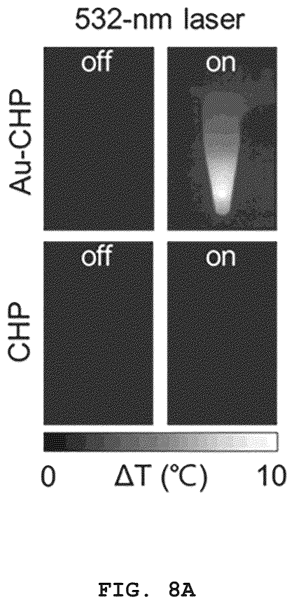

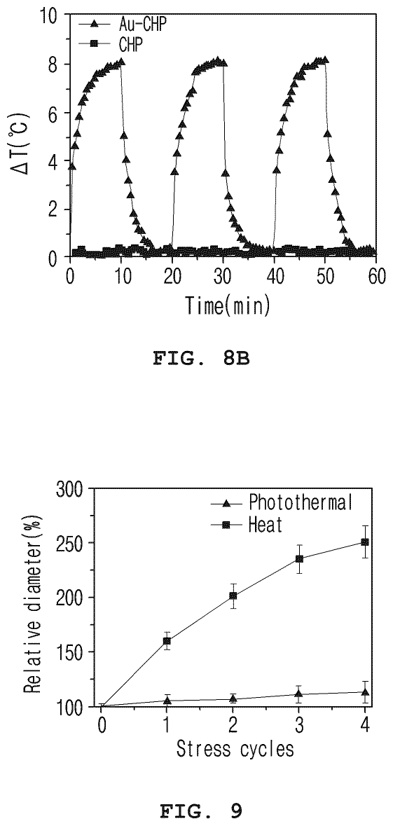

Direct heating of the solution is not suitable for protein delivery because of protein denaturation at high temperatures. Therefore, localized heat generation by light (i.e., photothermal conversion) may be an alternative way to release proteins from the thermoresponsive moiety of Au-CHP. The maximum absorbance of Au-CHP occurred at 520 nm, and the illumination of the Au-CHP colloidal solution with resonant light resulted in a rapid temperature increase (ΔT=8.5° C.) in just one minute (FIGS. 8A and 8B).

The lyophilized Au-CHP powder showed a higher level of heat generation because Au-CHP is in a condensed state. However, no heat was generated in the CHP without AuNP. This indicates that the photothermal conversion effect of AuNP can occur in a local environment and induce a conformational change of the thermoresponsive PNIPAM structure of Au-CHP.

To verify the thermal stress endurance of Au-CHP, size recovery was tested through multiple cycles of thermal and light stimulation. As shown in FIG. 9, under the photothermal conversion by light, the hydrodynamic diameter of Au-CHP remained stable, whereas the diameter of Au-CHP in the directly heated solution increased rapidly. Protein delivery with high stability can be achieved by utilizing the structural change of Au-CHP induced by photothermal heating.

### Experimental Example 5: Analysis of Protein Loading Efficiency and Surface Charge of Au-CHP

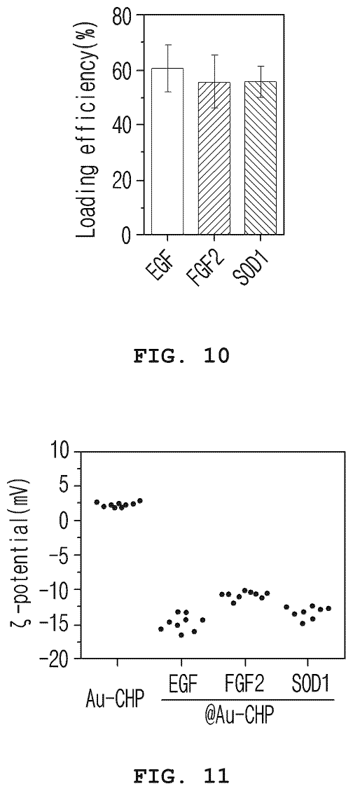

Au-CHP was loaded with Ara27 CPP conjugated proteins (e.g. Ara27-FGF2, Ara27-SOD1, and Ara27-EGF), and the protein loading efficiency of Au-CHP was calculated. The loading efficiencies of the three proteins in Au-CHP were 60.55%±8.32% (EGF), 55.63%±9.65% (FGF2), and 57.65%±5.66% (SOD1), respectively, with similar results for all three proteins (FIG. 10). Looking at the change in surface charge before and after protein loading (FIG. 11), it is believed that the proteins were bound through electrostatic interactions.

### Experimental Example 6: Protein Release Test of Protein-Loaded Au-CHP after Light Irradiation

The cumulative protein release of Au-CHP and CHP was compared by irradiating with 532 nm light for 30 min (FIG. 12). The photothermal conversion of AuNP and the subsequent phase transition of the adjacent PNIPAM network caused the loaded protein to be released from Au-CHP. As a result, the surface charge of Au-CHP recovered to a positive (+) value after protein release, showing high reversibility.

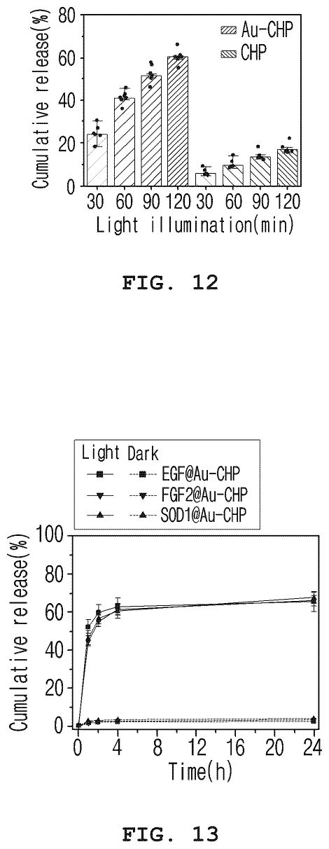

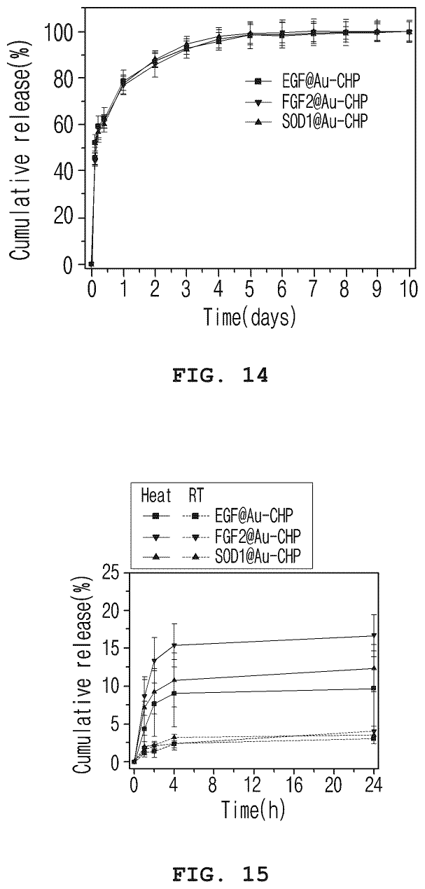

After 24 hours, more than 60% of the loaded protein was released, regardless of molecular weight, and complete release occurred within 5 days, whereas no protein release was observed in CHP in the absence of light (FIG. 13 and FIG. 14).

In addition, the cumulative release of proteins was much higher during photothermal conversion than when responding to heat. As shown in FIG. 15, the cumulative release amount when heat was applied was within 15%. These results demonstrate that the structural changes in Au-CHP through local light stimulation lead to an efficient release of the protein.

### Experimental Example 7: Delivery Test of FGF2 Protein Loaded in Au-CHP

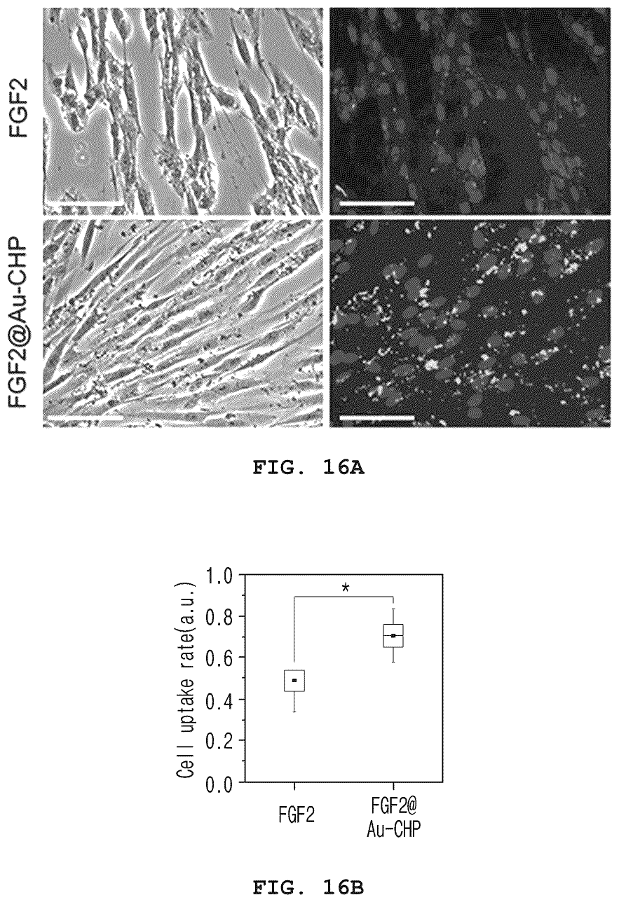

As shown in FIGS. 16A and 16B, it was confirmed that the cellular delivery of FGF2 was improved over a wide area when loaded in Au-CHP.

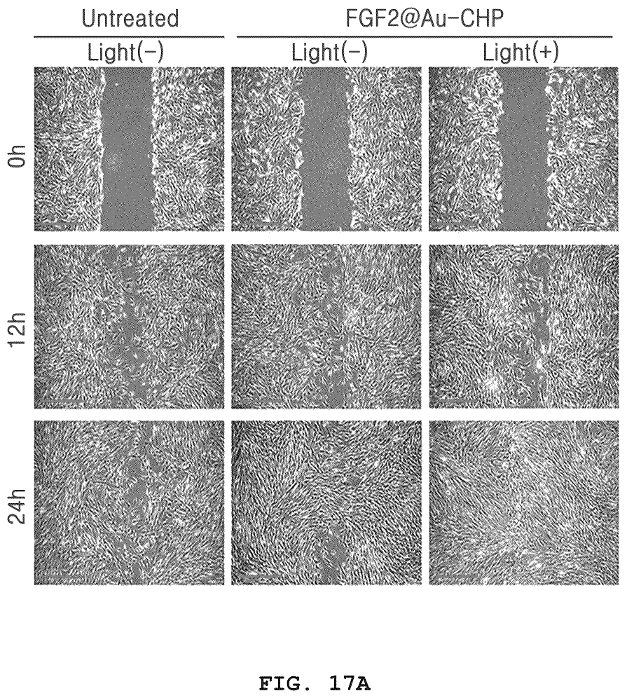

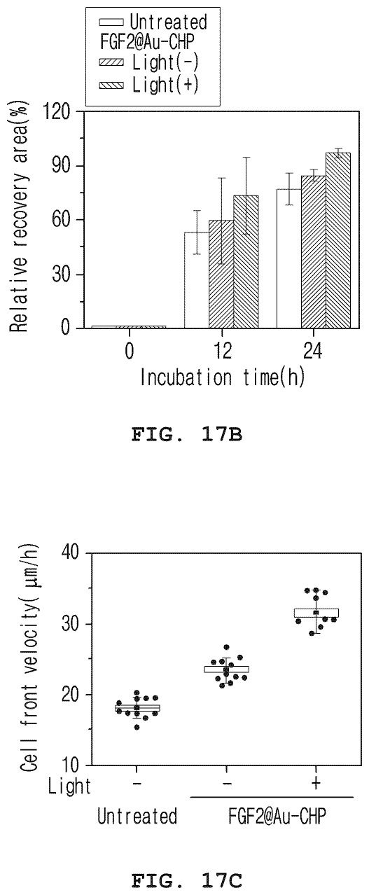

To determine the cellular activity of the delivered FGF2, a wound-healing assay was performed using human dermal fibroblasts (HDF) (FIG. 17A).

HDF in suspension was seeded on 18 mm glass coverslips and incubated for 24 hours, and then a scratch assay was performed. After washing twice with PBS, wound-mimicking scratches were created using a sterilized 200 μL pipette tip. Cells were cultured overnight in a medium containing FGF2-loaded CHP or Au-CHP, and samples were examined for confluent cell monolayers using a Leica DMi8 inverted microscope. Finally, time-lapse images were used to confirm the recovery of the empty wound area and analyzed quantitatively using ImageJ software.

When treated with FGF2@Au-CHP, the recovery area (FIG. 17B) and cell front velocity (FIG. 17C) of the stimulated cells significantly increased compared to the untreated group after 24 hours, regardless of light exposure, and the cell front velocity of the light-exposed group was significantly higher than that of the other groups.

### Experimental Example 8: Delivery Test of SOD1 Protein Loaded in Au-CHP

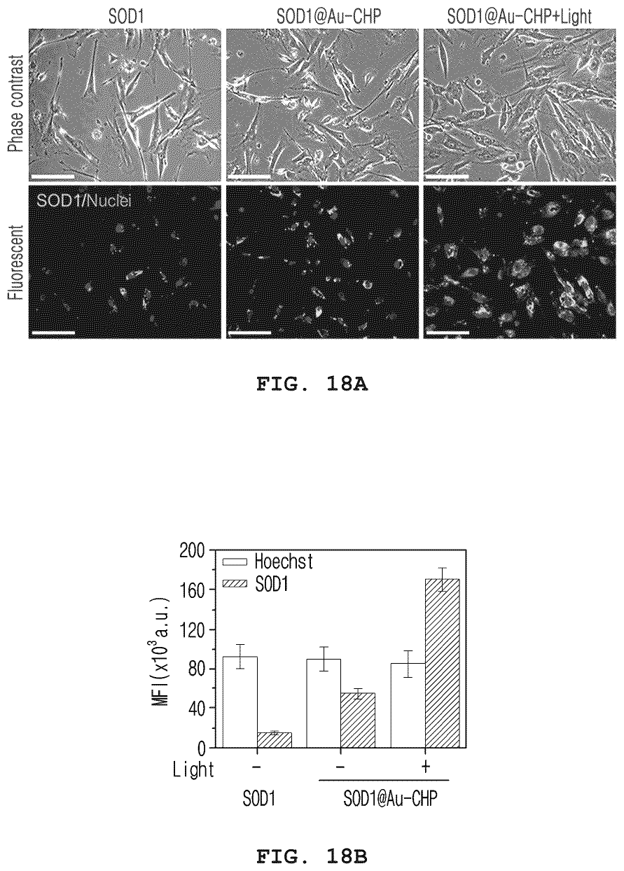

The effectiveness of enzyme protein delivery was tested using Au-CHP loaded with SOD1 (SOD1@Au-CHP). SOD1 is known as a cellular defense system that effectively removes reactive oxygen species (ROS). As shown in FIG. 18A, after photothermal treatment with SOD1 @Au-CHP, the fluorescence signal of SOD1 in the cytoplasm was significantly increased, whereas intracellular SOD1 accumulation was minimal in both the case of SOD1 alone (i.e., negative control) and the absence of light exposure (i.e., positive control). For quantitative analysis of the delivered SOD1, mean fluorescence intensity (MFI) per cell was used. The MFI values of the group exposed to SOD1 @Au-CHP were significantly increased by approximately 5.4-fold and 8.2-fold compared to the positive and negative controls, respectively (FIG. 18B). The scattered spots corresponding to Au-CHP were evenly distributed in the cytoplasm near the nucleus, and in particular, the signals from the clustered spots were confirmed to overlap with the fluorescence signal of the loaded protein. Therefore, it was confirmed that SOD1 released from Au-CHP upon stimulation effectively removed active oxygen species in the cytoplasm.

To evaluate the antioxidant activity, SOD1@Au-CHP (1 μg/mL SOD1) was applied to reactive oxygen species (ROS)-induced human dermal fibroblasts treated by incubation with 1 μM H₂O₂ overnight. Each sample was evaluated under illuminated and non-illuminated conditions for 30 minutes to initiate protein release. The activity of the released SOD1 was analyzed by observing the fluorescence signal of reactive oxygen species using dichlorofluorescein diacetate fluorescence assay (DCFDA assay) (FIG. 19).

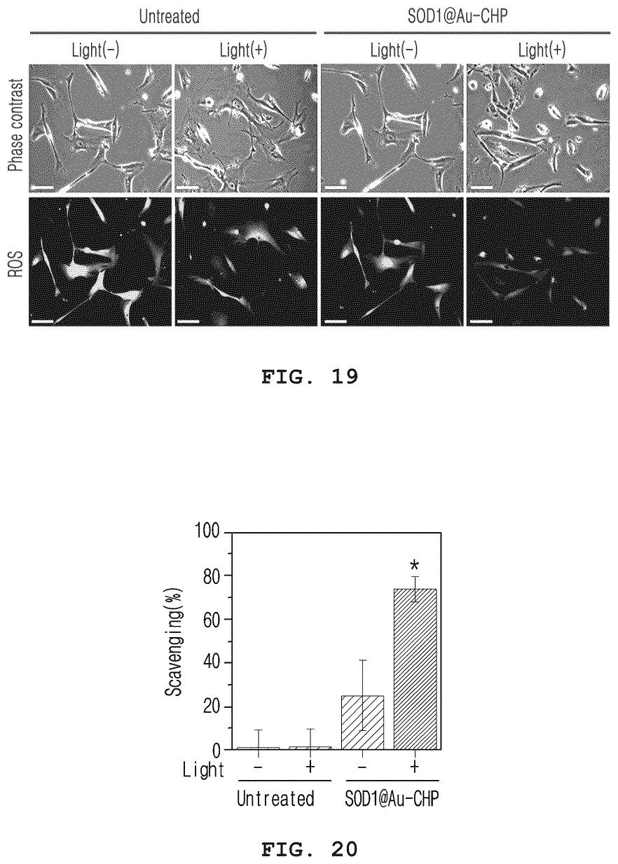

The group treated with SOD1@Au-CHP showed a 73.3% decrease in intracellular ROS levels, whereas the untreated group showed high levels of fluorescence regardless of the presence or absence of light. The SOD1 @Au-CHP group that was not exposed to light also showed a significant decrease in ROS levels with relatively high fluctuation ranges (FIG. 20), indicating that the outermost protein in the Au-CHP group can regulate the levels of cytosolic ROS. These results suggest that the proteins photothermally released from Au-CHP do not lose their activity, demonstrating the excellent regulating properties of Au-CHP to control protein release kinetics in an on-demand manner.

### Experimental Example 9: Cellular Permeability Test of EGF Protein Loaded in Au-CHP

To validate the enhanced protein permeability using Au-CHP, the penetration depth was monitored using human dermal fibroblast (HDF) spheroids over 500 μm in diameter.

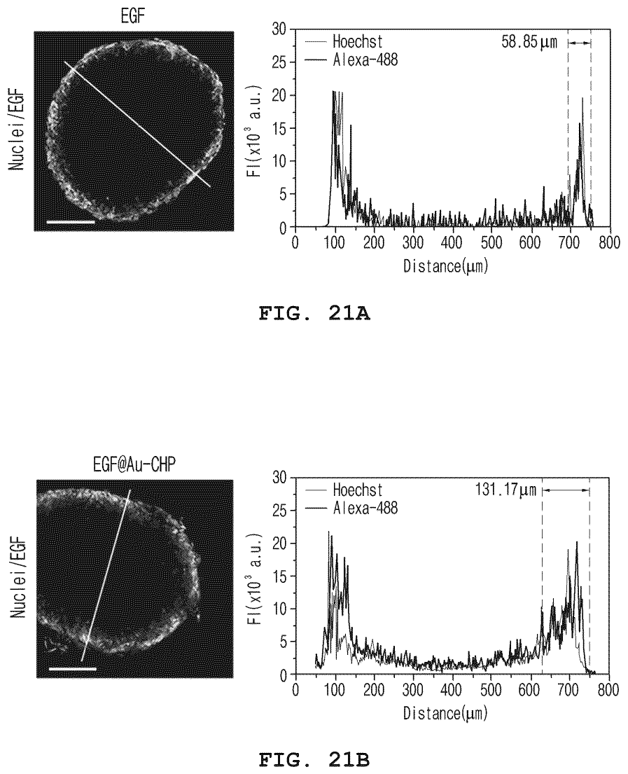

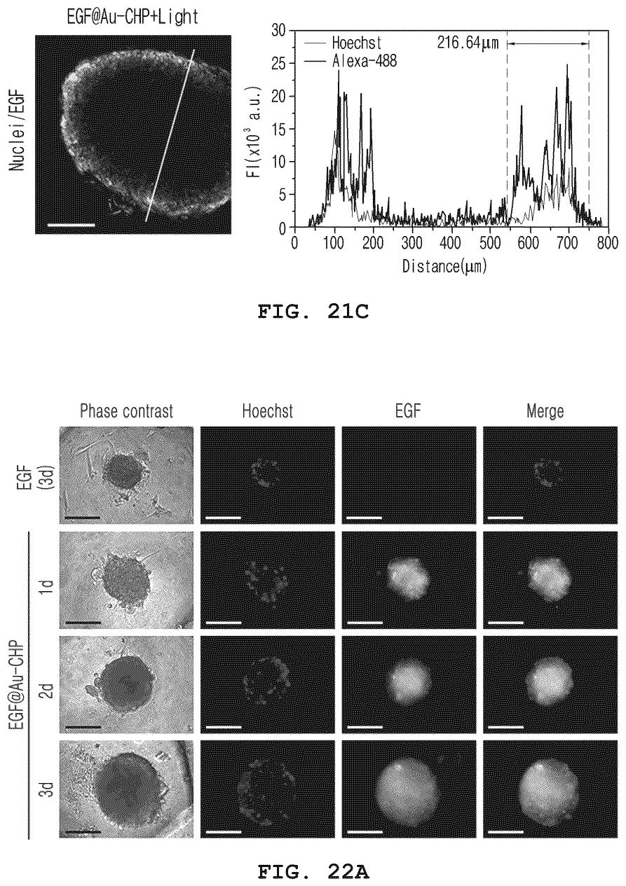

Spheroids were cultured with EGF alone and EGF@Au-CHP and then illuminated with light (532 nm laser, 3.5 W/cm²). An additional 6 hours of incubation allowed the released protein to diffuse further into deeper regions of the spheroids. As shown in FIG. 21A, FIG. 21B and FIG. 21C, Confocal images at the center plane of the spheroid showed that most of the signals corresponding to nuclei and EGF overlapped at the outer boundary of the spheroid. For a detailed comparison, the depth profiles of the overlapping fluorescence signals were examined. Under light-free conditions, the penetration depth of EGF using Au-CHP was increased to 2.11 times (i.e. 131 μm) compared to the negative control (i.e. 58 μm). This may be due to the synergistic effect of collagen and cell-inducing proteins, as it has been previously reported that collagen can be grafted into cargo carriers to improve biocompatibility as well as penetration into skin tissues. In particular, the penetration depth by photothermal treatment of EGF@Au-CHP was increased by 3.68-fold (i.e., up to 216.64 μm), indicating that the released protein can easily be involved in cell-cell interactions.

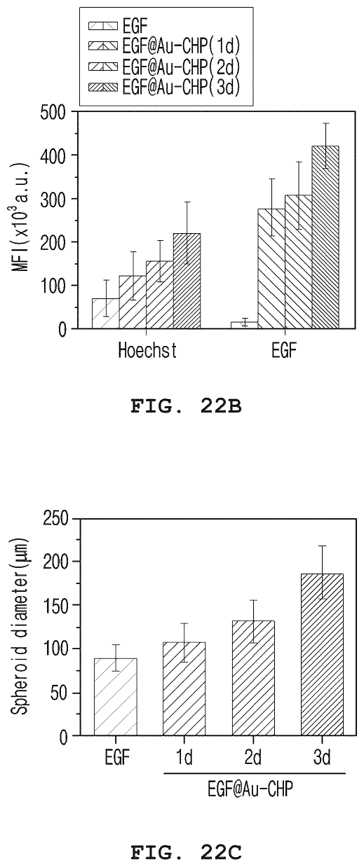

As shown in fluorescence images of FIG. 22A, the spheroids treated with Au-CHP loaded with EGF retained the fluorescence signal of EGF after 3 days of culture, while the spheroids treated with EGF alone showed no signal after 3 days of culture. Thus, by retaining the protein inside the intracellular spheroids after photothermally induced release using Au-CHP, the protein could be successfully delivered into the cells and accumulated continuously (FIG. 22B), which leads to a dramatic increase in spheroid size. When EGF was delivered using Au-CHP, the diameter of the spheroids increased by 189% compared to the spheroids treated with unencapsulated EGF (i.e., spheroids treated with EGF alone) (FIG. 22C).

### Experimental Example 10: Determination of Toxicity of Au-CHP (Biocompatibility)

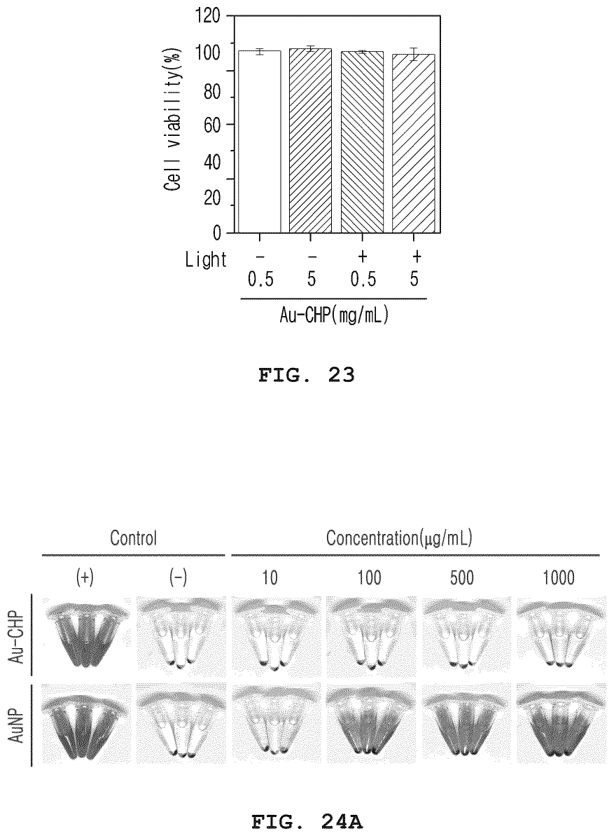

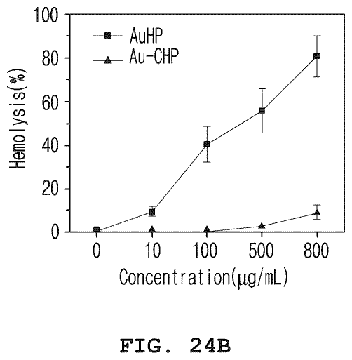

Before testing intracellular protein delivery using Au-CHP, cell viability was assessed to determine whether Au-CHP would cause toxicity when exposed to light. As shown in FIG. 23, FIG. 24B and FIG. 24C, Au-CHP exhibited excellent biocompatibility and minimal hemolysis, and the integration of polymer networks could effectively alleviate cytotoxicity and red blood cell (RBC) toxicity associated with high concentrations of pure AuNP. This biocompatibility was confirmed even at high-concentration treatment (5 mg/mL). In particular, it was shown that photoresponsive Au-CHP could be used to promote protein release with minimal cell damage, as it did not negatively affect cell survival even after exposure to light.

Having now fully described the present invention in some detail by way of illustration and examples for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

When a group of materials, compositions, components or compounds is disclosed herein, it is understood that all individual members of those groups and all subgroups thereof are disclosed separately. Every formulation or combination of components described or exemplified herein can be used to practice the invention, unless otherwise stated. Whenever a range is given in the specification, for example, a temperature range, a time range, or a composition range, all intermediate ranges and subranges, as well as all individual values included in the ranges given are intended to be included in the disclosure. Additionally, the end points in a given range are to be included within the range. In the disclosure and the claims, “and/or” means additionally or alternatively. Moreover, any use of a term in the singular also encompasses plural forms.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements.

One of ordinary skill in the art will appreciate that starting materials, device elements, analytical methods, mixtures and combinations of components other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. The invention illustratively described herein suitably may be practiced in the absence of any element or elements, limitation or limitations which is not specifically disclosed herein. Headings are used herein for convenience only.

All publications referred to herein are incorporated herein to the extent not inconsistent herewith. Some references provided herein are incorporated by reference to provide details of additional uses of the invention. All patents and publications mentioned in the specification are indicative of the levels of skill of those skilled in the art to which the invention pertains. References cited herein are incorporated by reference herein in their entirety to indicate the state of the art as of their filing date and it is intended that this information can be employed herein, if needed, to exclude specific embodiments that are in the prior art.

What is claimed is:

1. A plasmonic metal-collagen hybrid nanoparticle for protein delivery, comprising a collagen of 1 kDa to 30 kDa, a thermosensitive polymer monomer, a plasmonic metal nanoparticle, and a photoinitiator.

2. The plasmonic metal-collagen hybrid nanoparticle according to claim 1, wherein the collagen is derived from marine organisms.

3. The plasmonic metal-collagen hybrid nanoparticle according to claim 1, wherein the collagen has a molecular weight of 3 kDa to 10 kDa.

4. The plasmonic metal-collagen hybrid nanoparticle according to claim 1, wherein the thermosensitive polymer monomer is selected from the group consisting of N-isopropylacrylamide (NIPAM), N-vinylcaprolactam, (dimethylamino)ethylmethacrylate, and N, N-dimethylacrylamide.

5. The plasmonic metal-collagen hybrid nanoparticle according to claim 1, wherein the plasmonic metal nanoparticle is contained in an amount of 1 to 20 volume %.

6. The plasmonic metal-collagen hybrid nanoparticle according to claim 1, wherein the loaded protein is released by irradiating the plasmonic metal-collagen hybrid nanoparticle with light.

7. The plasmonic metal-collagen hybrid nanoparticle according to claim 1, wherein the plasmonic metal is selected from the group consisting of gold, silver, platinum, aluminum, or copper.

8. The plasmonic metal-collagen hybrid nanoparticle according to claim 7, wherein the plasmonic metal is gold.

9. A protein delivery vehicle comprising the plasmonic metal-collagen hybrid nanoparticle of claim 1.

10. A method for preparing a plasmonic metal-collagen hybrid nanoparticle for protein delivery, comprising a step of irradiating a mixture containing collagen of 1 kDa to 30 kDa, a thermosensitive polymer monomer, a plasmonic metal precursor, and a photoinitiator with ultraviolet light.
